(19) United States
(12) Patent Application Publication
(10) Pub. No.: US 2011/0184468 A1
(43) Pub. Date: Jul. 28, 2011

(54) SPINOUS PROCESS FUSION PLATE WITH OSTEOINTEGRATION INSERT

(75) Inventors: Newton H. Metcalf, JR., Memphis, TN (US); Gregory C. Marik, Collierville, TN (US); Nicholas M. Benson, Cordova, TN (US)

(73) Assignee: Warsaw Orthopedic, Inc., an Indiana Corporation, Warsaw, IN (US)

(21) Appl. No.: 12/695,508

(22) Filed: Jan. 28, 2010

(51) Int. Cl. A61B 17/88; A61B 17/80

(52) U.S. Cl. 606/279; 606/70; 606/297

(57) ABSTRACT

A spinal implant clamps to the spinous processes and provides an osteointegration material between the spinous processes so that there may be osteointegration between the spinous processes and a portion of the implant located in the interspinous space. The implant includes first and second plates, a post that extends between the plates and into at least one of the plates, a locking mechanism for locking the relative positions of the plates, and an osteointegration sleeve. The sleeve is disposed between the first and second plates and has an inner surface defining a longitudinal through-passage that is disposed about an outer surface of the post. The sleeve includes osteointegration material and is distinct from the post. The osteointegration material of the sleeve may be, for example, allograft bone or a bone growth material such as bone morphogenetic protein. Related methods are also described.

### BACKGROUND

The present application is directed to a spinal stabilization device that is attached to adjacent spinous processes and provides a means for osteointegration therebetween.

Spinal stabilization techniques may include the affixation of a plate to adjacent spinous processes. For example, a spinal implant sold under the trade name CD HORIZON SPIRE™ by Medtronic Spinal and Biologics of Memphis, Tenn. may be attached to adjacent spinous processes in a clamping fashion so as to stabilize the associated vertebral bodies, typically as part of a spinal fusion procedure. The conventional CD HORIZON SPIRE™ product provides stabilization by clamping to the spinous processes to fix their relative positions, and thereby provides support for fusion devices that may be installed elsewhere on the associated spinal motion segment. However, the conventional CD HORIZON SPIRE™ product does not itself provide a bone growth path between the spinous processes due to the materials and configuration thereof.

While such stabilization devices are suitable for many situations, there remains a need for alternative spinal stabilization devices, particularly ones that advantageously include an osteointegration path for use in spinal fusion applications.

### SUMMARY

The present application is directed to a spinal implant that clamps to the spinous processes and provides an osteointegration material between the spinous processes so that there may be osteointegration between the spinous processes and a portion of the implant located in the interspinous space.

In one embodiment, the present invention provides a spinal implant comprising: a first plate having a medial face configured to abut adjacent spinous processes and a second plate having a medial face configured to abut the adjacent spinous processes. The second plate is disposed in spaced relation to the first plate. A post extends along a longitudinal post axis oriented transverse to the medial face of the second plate. The post extends from the first plate through at least a portion of the second plate so as to interconnect the first and second plates. The medial faces of each of the plates extend from the post in generally opposite directions therefrom between a superior end of the respective plate that is positionable along a superiorly located spinous process and an inferior end of the respective plate that is positionable along an inferiorly located spinous process. An osteointegration sleeve comprises an osteointegration material and is distinct from the post. The sleeve is disposed between the first and second plates and has an inner surface defining a longitudinal through-passage that is disposed about an outer surface of the post. A locking mechanism is associated with the second plate. The second plate is moveable along the post toward the first plate from a first position to second position. The second plate is lockable relative to the first plate via engagement of the locking mechanism with the post. The sleeve is advantageously slidable along the post when the second plate is in the first position. The osteointegration material of the sleeve may, in some embodiments, be allograft bone or a bone growth material such as bone morphogenetic protein.

The present invention also provides a method of stabilizing adjacent spinous processes. In one embodiment, the method comprises positioning a first plate along a first side of the adjacent spinous processes such that a medial face of the first plate is oriented toward the spinous processes; disposing a post between the spinous processes so as to extend through a sagittal plane defined thereby; the post having a longitudinal post axis disposed transverse to the medial face of the first plate; optionally disposing an osteointegration sleeve about the post so as to be slidable along the post; the sleeve distinct from the post and comprising an osteointegration material; positioning a second plate along a second side of the adjacent spinous processes such that a medial face of the second plate is oriented toward the spinous processes; the second plate disposed such that a portion of the post extends through the medial face of the second plate with the sleeve disposed between the first and second plates. Thereafter, the method comprises clamping the spinous processes between the first and second plates by moving the second plate toward the first plate along the post axis from a first position to a second position; and locking the second plate in the second position such that the sleeve is disposed to extend through the sagittal plane with the sleeve abutting both of the adjacent spinous processes.

The various aspects of the various embodiments disclosed herein may be used alone or in any combination, as is desired.

### BRIEF DESCRIPTION OF THE DRAWINGS

FIG. 1 shows a implant according to one embodiment of the present invention installed between the L4 and L5 vertebrae.

FIG. 2 shows a perspective view of the implant of FIG. 1.

FIG. 3 shows a posterior view of the implant of FIG. 1 with the locking screw removed and the integration sleeve in phantom lines for illustration purposes, and the second plate in the second (clamped) position.

FIG. 4 is similar to FIG. 3, with the second plate in the first (unclamped) position.

FIG. 5 shows one embodiment of an integration sleeve suitable for one or more embodiments of the present invention.

FIG. 6 shows another embodiment of an integration sleeve suitable for one or more embodiments of the present invention.

FIG. 7 shows a posterior view of an implant according to another embodiment after implantation.

### DETAILED DESCRIPTION

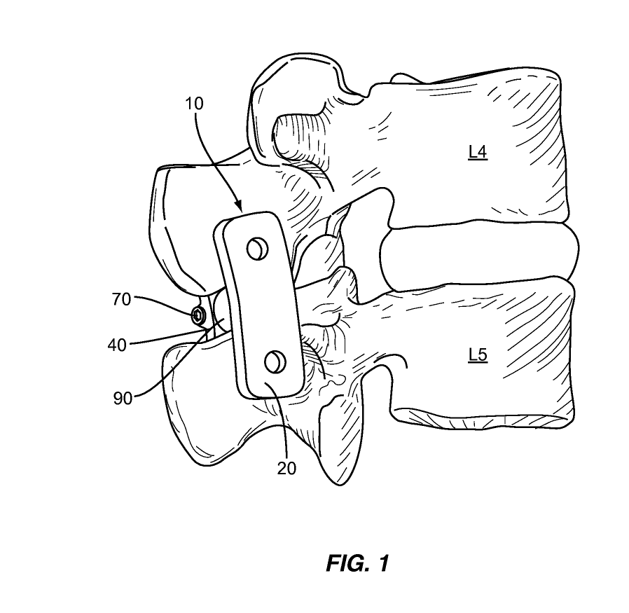

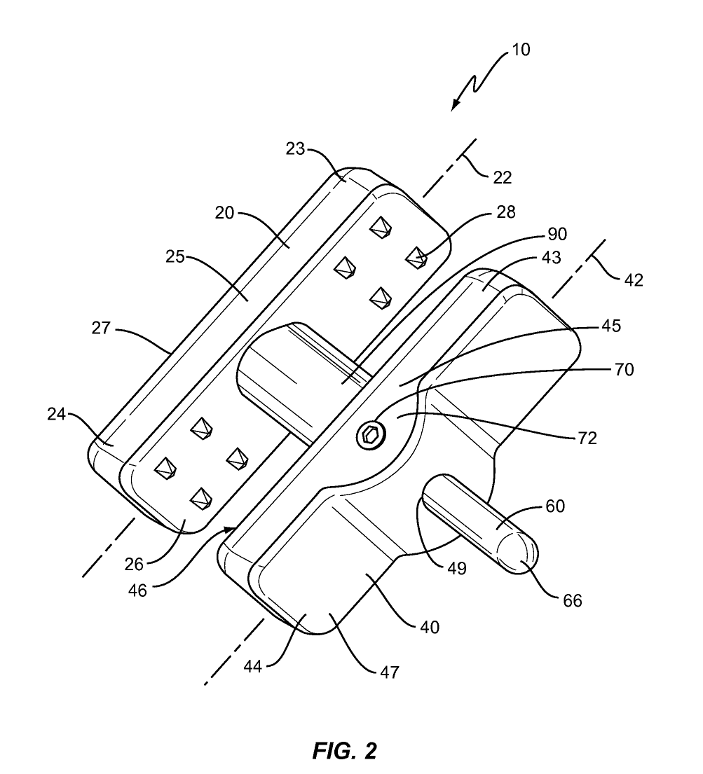

The present application is directed to a spinal implant that clamps to adjacent spinous processes SP1,SP2 and provides an osteointegration path therebetween. In one embodiment, shown in FIGS. 1-4, the implant, generally indicated at 10, includes a first plate 20, a second plate 40, an interconnecting post 60, a locking member 70, and an integration sleeve 90. The first plate 20 may be elongate along an associated longitudinal axis 22, with a superior end 23, an inferior end 24, and an intermediate section 25. The first plate 20 has a length sufficient to vertically span an interspinous gap between adjacent spinous processes while substantially overlapping the spinous processes. The first plate 20 has a medial face 26 and an opposite lateral face 27. The medial face 26 advantageously includes a one or more grip-enhancing surface features 28 for enhancing grip of the spinous processes SP1,SP2. Examples of grip-enhancing surface features 28 include ridges, knurlings, teeth, etchings, and the like. Advantageously, the grip-enhancing features take the form of a plurality of teeth or other projections that extend medially therefrom for biting into the spinous processes. The teeth 28 may advantageously be disposed in two groups, one on the superior end and one on the inferior end, with the intermediate section 25 being free of such teeth. The lateral face 27 may have suitable features, such as recesses or the like, for cooperating with installation and manipulation instrumentation. The superior end 23 and inferior end 24 are advantageously generally rounded so as to minimize damage to surrounding tissue and for ease of installation.

The second plate 40 may be substantially similar to the first plate 20. For example, the second plate 40 may be elongate along an associated longitudinal axis 42, with a superior end 43, an inferior end 44, and an intermediate section 45. The second plate 40 advantageously has a length sufficient to vertically span the interspinous gap while substantially overlapping the spinous processes. The second plate 40 has a medial face 46 and an opposite lateral face 47, with the medial face 46 facing the medial face 26 of the first plate 20. The medial face 46 advantageously includes grip-enhancing surface features 48, as discussed above, for enhancing grip of the spinous processes. The lateral face 47 may have suitable features, such as recesses or the like, for cooperating with installation and manipulation instrumentation. The superior end 43 and inferior end 44 are advantageously generally rounded so as to minimize damage to surrounding tissue and for ease of installation. The intermediate section 45 of the second plate 40 advantageously includes a through hole 49 that extends generally perpendicular to the longitudinal axis 42 and is sized to slidingly receive the post 60. The lateral face 47 in the intermediate section 45 may have a suitable boss or protrusion 72 thereon, with a hole 74 for receiving the locking member 70, as discussed below.

The post 60 of FIG. 1 takes the form of a simple round shaft that extends along a post axis 62 from a proximal end 64 to a distal end 66. The post 60 has a length sufficient to extend laterally across the interspinous gap, through the medial face 46 of the second plate 40, and into engagement with the fastener 70. In some embodiments, the post proximal end 62 is mounted to the first plate 20 by any suitable means, such as welding or the like. Alternatively, the post 60 may be integrally formed with the first plate 20. Still further, the post 60 may be pivotally attached to the first plate 20. The post distal end 66 may include flats or other features (not shown) for engaging with the locking member 70. The post 60 may advantageously be solid rather than hollow, and therefore typically will not have any longitudinal hollow inner cavities.

The locking member 70 of FIG. 1 takes the form of a simple setscrew that is sized to threadably engage hole 74 in second plate 40. When tightened, the locking member 70 presses against the post 60 to lock the relative distance between the plates 20,40.

As will be appreciated, the first plate 20, second plate 40, post 60, and fastener 70 are made from any suitable biocompatible rigid materials such as titanium and its alloys, stainless steel, ceramics, relatively rigid polymers, or the like, known in the art.

The integration sleeve 90, in one embodiment, takes the form of a generally cylindrical element having an outer surface 92 and an inner surface 94. The inner surface 94 defines a longitudinal through-passage or bore 96 that extends from the proximal face 98 to the distal face 99 of the integration sleeve 90. The bore 96 is configured to slidably receive the post 60; as such, the cross-section of the bore 96 is advantageously slightly larger than, and disposed outside of, the corresponding portion of the post 60. The integration sleeve 90 is advantageously relatively stiff such that the bore 96 is of a pre-defined shape. If desired, the bore 96 and post 60 may have suitable anti-rotation features, such as corresponding flats, corresponding ribs and grooves, or the like to prevent relative rotation between the integration sleeve 90 and the post 60.

The integration sleeve 90 has an outer dimension that is intended to vertically fill the interspinous gap. Thus, it is intended that the outer surface 92 of the integration sleeve 90 will abut the spinous processes SP1,SP2 upon device installation. As such, upon installation, the sleeve 90 extends between and contacts spinous processes SP1,SP2 to maintain or provide post-operative distraction between the spinout processes SP1,SP2.

The integration sleeve 90 provides an osteointegration path between the spinous processes SP1,SP2. The integration sleeve 90 comprises an osteointegration material. As used herein, osteointegration material means bone or bone growth material. Examples of bone include allograft bone or autograft bone. Specific examples of bone growth materials 100 include bone morphogenetic protein (BMP), various types of cement, transforming growth factor β1, insulin-like growth factor 1, platelet-derived growth factor, fibroblast growth factor, LIM mineralization protein, and/or other bone growth promotion materials known in the art. The integration sleeve may be formed entirely of osteointegration material, or may consist essentially of osteointegration material, or may be formed partially of osteointegration material. Thus, the sleeve may be formed, for example, entirely of allograft bone. In other embodiments, the integration sleeve 90 may be formed of a metal material with suitable passages 97 formed therein transverse to the bore 96 that contain bone growth material 100. In some embodiments, the integration sleeve 90 may be formed of a mesh or porous material, with bone growth material 100 disposed therein.

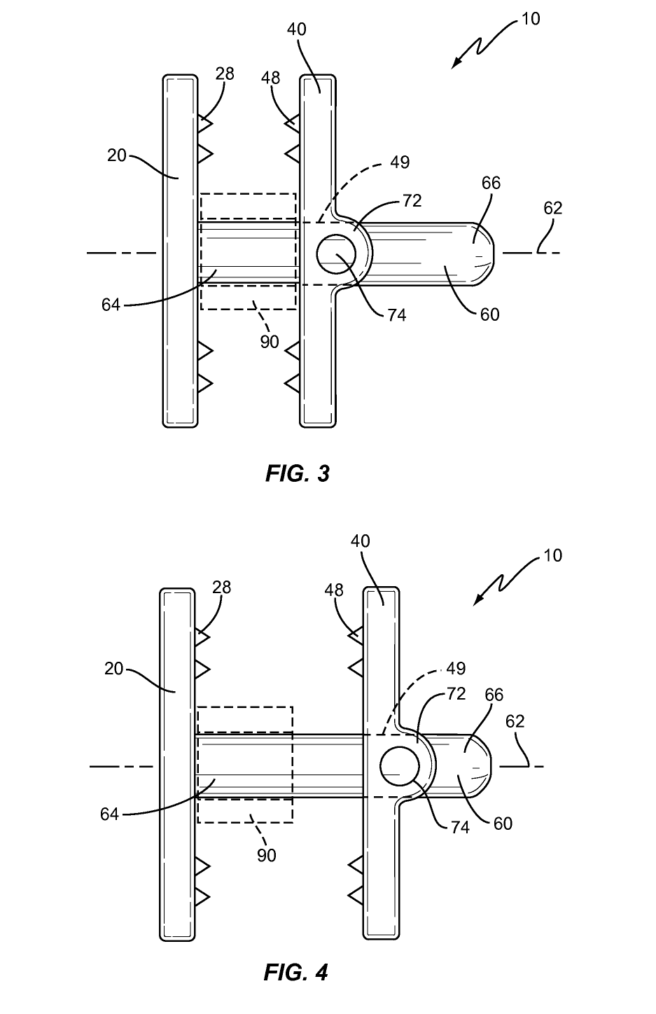

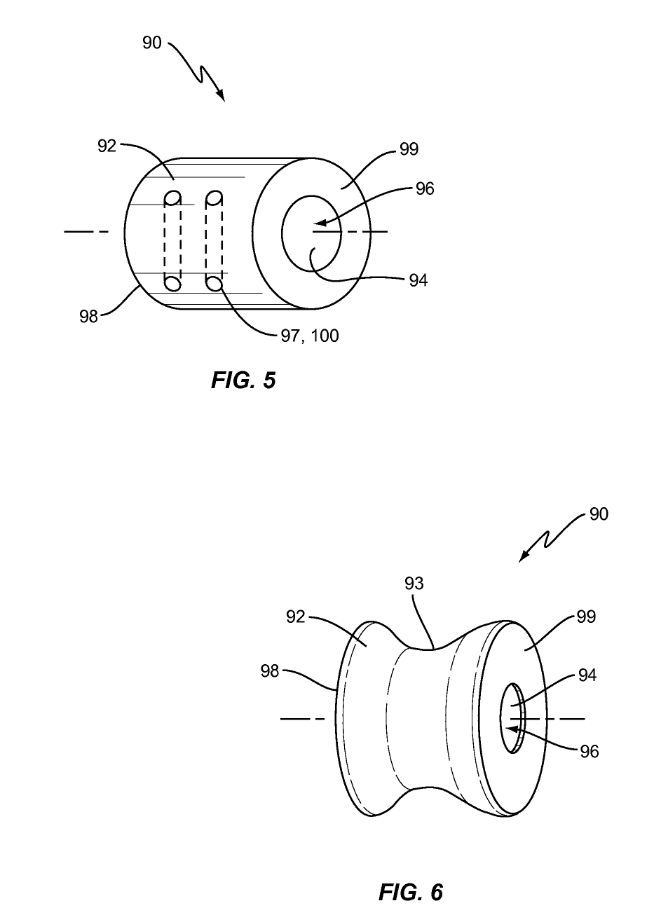

In use, the device 10 can be implanted for posterior spinal stabilization as a stand-alone procedure or in conjunction with other procedures. The device 10 can be positioned through a small posterior incision in the patient of sufficient size to admit the device and instrumentation. Following the incision, muscle is moved aside if and as needed for placement of the device 10 into position between spinous processes SP1, SP1. The spinous processes SP1,SP1 are optionally distracted using suitable instrumentation known in the art, and the device 10 is implanted such that the integration sleeve 90 is positioned between the spinous processes SP1,SP2 and the plates 20,40 extend on respective lateral sides thereof. In some embodiments, the device 10 may be inserted in an already-assembled condition, with the second plate 40 disposed on the post 60, with the device being either assembled by the manufacturer or by medical personnel. In other embodiments, the device 10 may be assembled in-situ. The locking member 70 can be loosened if necessary and the plates 20,40 pushed toward one another with a compression instrument or manually, to move (e.g., slide) the second plate 40 along the post 60 toward the first plate 20 from a first position (FIG. 3) farther from the first plate 20 to a second position (FIG. 4) closer to the first plate 20. This movement causes the plates 20,40 to clamp the spinous processes SP1,SP2. If spikes 28,48 are provided, compression is advantageously continued until the spikes 28,48 are sufficiently engaged to the bony material of the spinous processes SP1,SP2. Advantageously, the sleeve 90 is sized such that the sleeve proximal face 98 abuts the medial face 26 of the first plate 20 and the sleeve distal face 99 abuts the medial face 46 of the second plate 40 once the plates 20,40 are fully clamped to the spinous processes. Following engagement of plates 20,40 on the spinal processes, locking member 70 is tightened onto post 60 using an appropriate instrument to lock the relative positions of the plates 20,40. If desired, locking member 70 may be provided with a break-off portion (not shown) that provides an indication when sufficient torque is applied.

Clamping plates 20,40 to the spinous processes SP1,SP2 helps maintain the alignment and spacing of the spinous processes SP1,SP2 while also providing resistance to spinal extension and flexion. Thus, engagement of plates 20,40 to the spinous processes SP1,SP2 resists movement of the spinous processes SP1,SP2 toward and away from one another as a result of spinal extension and flexion, respectively, or as a result of any other movement or condition. Integration sleeve 90 extends between plates 20,40 and also between spinous processes SP1,SP2 to resist movement of the spinous processes toward one another as a result of spinal extension. Integration sleeve 90 thus provides support of the vertebrae to maintain or provide post-operative distraction between the spinous processes SP1,SP2.

It is intended that there will be osteointegration of the integration sleeve 90 over time. The osteointegration may occur as the bone from the spinous processes SP1,SP2 grows into pores on the outer surface of the sleeve 90, and/or as the bone grows through any bone passages 97 in sleeve 90. This osteointegration should result in further anchoring the implant device 10 between the spinous processes SP1,SP2.

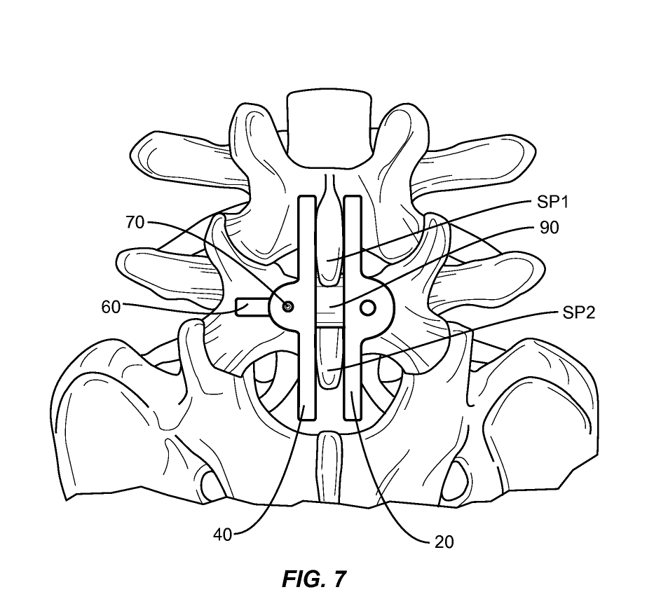

The embodiment of FIG. 1 includes a post 60 that is fixed relative to the first plate 20. However, in some embodiments, the post 60 is moveable relative to the first plate 20. For example, the post 60 may be pivotally coupled to the first plate 20 (see FIG. 7), either for mono-axial or poly-axial movement relative to the first plate 20 about one or more pivot axes, such as about a pivot axis perpendicular to the post axis 62 and parallel to medial face 26. Examples of suitable pivoting structures are shown in U.S. Pat. No. 7,048,736 and U.S. Patent Application Publications 2006/0247640, both of which are incorporated herein in their entirety. In addition, while the plates 20,40 of FIG. 1 are straight, and thus their longitudinal axes 22,42 are straight lines, such is not required. In some embodiments, it may be advantageous for the plates 20,40 to have a curved longitudinal axis 22,42 so as to better match spinal lordosis. Thus, the perimeter edge of the plates 20,40 may have a concavo-convex shape in some embodiments.

In addition, while the integration sleeve 90 has been discussed above in the context of a hollow cylindrical body, such is not required. Indeed, the integration sleeve 90 may have a non-circular cross-sectional perimeter, such as an oval, round or oval with flats, semi-rectangular, star-shaped, or any other suitable shape. Further, the outer surface 92 of integration sleeve 90 may have one or more circumferential grooves 93 or the like, and/or may otherwise be longitudinally concavely shaped. For example, the integration sleeve 90 may have a generally H-shape in cross-section, with the cross-member of the H being generally parallel to the post axis Likewise, the integration sleeve 90 may have a non-uniform thickness when viewed in a posterior-to-anterior direction (when installed), advantageously thinner in the posterior direction and thicker in the anterior direction. The proximal face 98 and distal face 99 may be normal to the longitudinal post axis 62 (when disposed on the post 60), or may be at a relative angle thereto, and either or both may be generally planar or have generally longitudinal projections and/or depressions. Further, while the integration sleeve 90 may fully circumscribe the post 60 (i.e. annularly surround); such is not required. Instead, there may be a circumferential section of the post 60, being less than half (e.g., 25%), that is left uncovered by the sleeve 90 so that the sleeve 90 has a C-shaped cross-section. Such a C-shaped sleeve 90 is considered to be disposed about the post 60 when the post 60 is received in the sleeve's bore 96. The integration sleeve 90 may have a combination of the various aspects disclosed herein. For example, the integration sleeve 90 may, in an exemplary embodiment, appear generally trapezoidal when viewed in a lateral direction, and generally H-shaped when viewed in a posterior-to-anterior direction, with the proximal face 98 being normal to the post axis 62 and the distal face 99 being at an angle thereto.

It is contemplated that any of the integration sleeves 90 can be provided in various sizes from which a desired size and/or shape can be selected by the surgeon. The integration sleeves 90 can be provided in a kit or as a set, and the sleeves 90 providing the desired outer surface profile and size may be selected by the surgeon to provide the desired contact or fit with the adjacent spinous processes based on the conditions learned of during pre-operative planning or encountered during surgery.

While FIG. 1 shows an implant applied to vertebra L-4 and L-5, the implant device can be implanted on spinous processes at other levels. Levels up to T-3 may be appropriate sites. Also, plates 20,40 bridging more than one level may also be considered, with multiple posts 60 and multiple sleeves 90 disposed at suitable intervals.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A spinal implant comprising:
a first plate having a medial face configured to abut adjacent spinous processes;
a second plate having a medial face configured to abut the adjacent spinous processes; said second plate disposed in spaced relation to said first plate;
a post extending along a longitudinal post axis oriented transverse to the medial face of the second plate; the post extending from the first plate through at least a portion of the second plate so as to interconnect the first and second plates;
wherein the medial faces of each of the plates extend from the post in generally opposite directions therefrom between a superior end of the respective plate that is positionable along a superiorly located spinous process and an inferior end of the respective plate that is positionable along an inferiorly located spinous process;
an osteointegration sleeve comprising an osteointegration material; the sleeve distinct from the post; the sleeve disposed between the first and second plates and having an inner surface defining a longitudinal through-passage that is disposed about an outer surface of the post;
a locking mechanism associated with the second plate;
wherein the second plate is moveable along the post toward the first plate from a first position to second position; wherein the second plate is lockable relative to the first plate via engagement of the locking mechanism with the post.

2. The implant of claim 1 wherein the osteointegration material of the sleeve is selected from the group consisting of bone morphogenetic protein, transforming growth factor β1, insulin-like growth factor 1, platelet-derived growth factor, fibroblast growth factor, and LIM mineralization protein.

3. The implant of claim 1 wherein the osteointegration material of the sleeve is allograft bone.

4. The implant of claim 1 wherein the sleeve consists essentially of osteointegration material.

5. The implant of claim 1 wherein the sleeve is slidable along the post when the second plate is in the first position.

6. The device of claim 1 wherein the second plate is both moveable along the post toward and away from the first plate and lockable relative thereto via engagement of the locking mechanism with the post at an infinite number of longitudinal positions along the post axis.

7. The implant of claim 1 wherein the sleeve through-passage has a predefined shape that corresponds to a cross-sectional shape of the post.

8. The implant of claim 1 wherein the post and the sleeve slidably interengage such that the sleeve is inhibited from rotating relative to the post about the post axis.

9. The implant of claim 1 wherein the sleeve has an outer surface with an outwardly concave profile parallel to the post axis.

10. The implant of claim 1 wherein the sleeve comprises a plurality of through passages disposed transverse to the post axis.

11. The implant of claim 1 wherein the sleeve completely circumscribes the post axis.

12. The implant of claim 1 wherein the medial face of at least one of the first plate and the second plate comprising a grip-enhancing surface feature.

13. The implant of claim 12 wherein the medial face of both the first plate and the second plate each comprise a plurality of inwardly projecting teeth.

14. A method for stabilizing adjacent spinous processes, comprising:
positioning a first plate along a first side of the adjacent spinous processes such that a medial face of the first plate is oriented toward the spinous processes;
disposing a post between the spinous processes so as to extend through a sagittal plane defined thereby; the post having a longitudinal post axis disposed transverse to the medial face of the first plate;
disposing an osteointegration sleeve about the post so as to be slidable along the post; the sleeve distinct from the post and comprising an osteointegration material;
positioning a second plate along a second side of the adjacent spinous processes such that a medial face of the second plate is oriented toward the spinous processes; the second plate disposed such that a portion of the post extends through the medial face of the second plate with the sleeve disposed between the first and second plates;
thereafter, clamping the spinous processes between the first and second plates by moving the second plate toward the first plate along the post axis from a first position to a second position;
locking the second plate in the second position such that the sleeve is disposed to extend through the sagittal plane with the sleeve abutting both of the adjacent spinous processes.

15. The method of claim 14 further comprising coupling the second plate to the post prior to the positioning the first plate along the first side of the adjacent spinous processes.

16. The method of claim 14 further comprising distracting spinous processes prior the clamping, and wherein the sleeve maintains at least a portion of the distraction.

17. The method of claim 14 further comprising selecting the osteointegration sleeve from a plurality of candidate sleeves that differ in at least one of material and dimensions.

18. The method of claim 14 wherein the osteointegration sleeve comprises an interior surface defining a longitudinal passage for receiving the post and at least one bone growth passage extending transverse thereto; wherein the bone growth passage contains bone growth material.

19. A method for stabilizing adjacent spinous processes, comprising:
positioning a first plate along a first side of the adjacent spinous processes such that a medial face of the first plate is oriented toward the spinous processes;
disposing a post between the spinous processes so as to extend through a sagittal plane defined thereby; the post having a longitudinal post axis disposed transverse to the medial face of the first plate;
positioning a second plate along a second side of the adjacent spinous processes such that a medial face of the second plate is oriented toward the spinous processes; the second plate disposed such that a portion of the post extends through the medial face of the second plate with the sleeve disposed between the first and second plates;
thereafter, clamping the spinous processes between the first and second plates by moving the second plate toward the first plate along the post axis from a first position to a second position;
locking the second plate in the second position such that:
the sleeve is disposed to extend through the sagittal plane with the sleeve abutting both of the adjacent spinous processes; and
an osteointegration sleeve is disposed about the post and captured between the plates, with the sleeve disposed between the post and the adjacent spinous processes and extending through the sagittal plane; the sleeve distinct from the post and comprising an osteointegration material.

20. The method of claim 19 wherein the second plate is coupled to the post during said positioning the first plate along the first side of the adjacent spinous processes.
